(19) United States
(12) Patent Application Publication
(10) Pub. No.: US 2005/0107285 A1
(43) Pub. Date: May 19, 2005

(54) Treatment of inflammatory conditions

(76) Inventors: Steven W Edwards, Liverpool (GB); Robert J Moots, Liverpool (GB)

Correspondence Address: PILLSBURY WINTHROP LLP, 725 S. FIGUEROA STREET, SUITE 2800, LOS ANGELES, CA, 90017, US

(21) Appl. No.: 10/495,377

(22) PCT Filed: Nov. 11, 2002
(86) PCT No.: PCT/GB02/05082

(30) Foreign Application Priority Data
Nov. 13, 2001 (GB) 0127206.1

(51) Int. Cl. A61K 38/00; A61K 31/60

(52) U.S. Cl. 514/2; 514/165

(57) ABSTRACT

The present invention relates to the use of agents that inhibit FcyRIIIb receptor activity, activation or expression for the treatment of inflammatory conditions such as Rheumatoid Arthritis. The invention also concerns screening methods that may be employed to identify said agents.

The present invention relates to the treatment of inflammatory conditions with receptor inhibitors and also methods of screening for agents that may have efficacy for treating such conditions.

Granulocytes, such as neutrophils, are essential components of the host defence against infections. Circulating granulocytes accumulate at sites of infection, where they are able to recognise and phagocytose microbial pathogens. The pathogens are eliminated by the activation and delivery of highly cytotoxic mediators directly into phagocytic vesicles within the granulocyte.

However, granulocytes can also be activated to secrete large quantities of such inflammatory mediators (e.g. highly toxic reactive oxygen intermediates (ROIs) and granule enzymes) into the extracellular environment. Such secretion of these highly toxic mediators can result in the development, or at least exacerbation, of an inflammatory condition and is likely to result in tissue damage.

Accordingly it may be considered that inhibition of granulocyte function may be an attractive means of decreasing inflammatory damage. However, to date, such a proposition has been discounted by those skilled in the art because the inhibition of granulocytes would also result in an unacceptable reduction in the host-defence response to invading pathogens By way of example, Rheumatoid Arthritis (RA) is an inflammatory condition characterised by inflamed synovial joints that lead to tissue damage and, ultimately, joint destruction. Of all the cells implicated in the pathology of RA, neutrophils have the greatest capacity to inflict tissue damage. Neutrophils heavily infiltrate inflamed synovial tissue in RA, particularly at the margin of the pannus and are the most abundant cell type in synovial effusions. Neutrophils have also been shown to play a key role in animal models of rheumatoid arthritis. They secrete large quantities of inflammatory mediators such as toxic reactive oxygen metabolites (such as O₂, H₂O₂, HOCl), granule enzymes (such as proteases, permeability inducing factors and defensins) and cytokines (including TNFα, IL-1β and IL-8). Neutrophils have previously been thought of as short-lived, terminally differentiated cells, dying rapidly by apoptosis. However, the inventors have shown that, at sites of inflammation, such as in the inflamed rheumatoid joint, neutrophils are able to live for extended periods of time and are capable of upregulating a variety of genes important in mediating inflammation. Accordingly granulocytes appear to play a significant role in the pathophysiology of inflammatory conditions.

The potential to decrease inflammatory damage in RA, by inhibiting the function of granulocytes such as neutrophils is attractive. However, such a proposition is not practical using conventional therapeutic agents. This is because the capacity for neutrophils to secrete toxic mediators in the joint must be decreased without compromising their effectiveness to phagocytose and kill pathogens.

Current therapies for RA are inadequate, both in their ability to adequately suppress disease activity and their unacceptable side effects. Treatment today can be considered as traditional (conventional) therapy and biologic therapy. “Traditional” drugs were, on the whole, discovered by serendipity, where a drug developed for a totally different condition was also found to be of benefit in RA. Biologic therapy currently comprises three drugs that, in contrast, have been developed as a result of a focused research programme into RA. These drugs are extremely expensive to manufacture and are delivered by injection. Manufacturing capacity for biologics cannot keep up with demand and indeed, the high cost of producing and administering these drugs (approx £9K per patient per annum) will further limit their widespread use. Both traditional and biologic therapies in RA are non-selective. In other words, the drugs are targeting either totally unknown molecules (as is the case with gold salts) or molecules which play an important role in RA (e.g. TNFα), but importantly, also crucial roles in normal life. As a consequence, the price paid for suppressing inflamed joints is that of side-effects, especially a weakening of the immune system or immunosuppression. The newest, biologic drugs were first hailed to be effective and safe. However, the data that is accruing from their usage in normal clinical practice shows that they are linked with a significant risk of serious infection, including death. In addition, biologics, together with some traditional anti-rheumatic drugs, also have a theoretical risk of inducing cancer.

There are currently four main groups of compound used to treat RA:
non-steroidal anti-inflammatory drugs (NSAIDs)
corticosteroids
Orally active “conventional” “disease-modifying” antirheumatic drugs (DMARDs)
biologics: such as anti-cytokine antibodies and cytokine receptor antagonists.

In the light of the above one of the objects of the present invention is to provide agents that may be used to inhibit extracellular secretion of toxic mediators from granulocytes (and thereby be useful for treating inflammatory conditions) without affecting phagocytosis and intracellular killing of invading pathogens by said granulocytes.

According to a first aspect of the present invention, there is provided a use of an agent which inhibits FcyRIIIb receptor activity, or activation or expression, for the manufacture of a medicament for the treatment of inflammatory conditions.

According to a second aspect of the present invention, there is provided a method for the treatment of inflammatory conditions comprising administering to a subject in need of such treatment a therapeutically effective amount of an agent which inhibits FcγRIIIb receptor activity, or activation or expression.

By “inflammatory condition” we mean a medical condition at least partially characterised by inappropriate secretion of inflammatory mediators (e.g. highly toxic reactive oxygen intermediates (ROIs) or granule enzymes or cytokines) from granulocytes into an affected tissue. Examples of such conditions include, but are not limited to, Rheumatoid Arthritis, Behcet's Disease, ANCA-associated vasculitis, systemic vasculitis, cystic fibrosis, asthma and Crohn's Disease.

By “FcγRIIIb receptors” we mean a granulocyte cell surface receptor which is a heavily-glycosylated protein of 50-70 kDa that is linked to the plasma membrane via an easily-cleaved glycosylphosphatidylinositol (GPI) anchor. Surface levels of FcγRIIIb are regulated by the balance between the rates of shedding and mobilisation of pre-formed intracellular stores to the cell surface. Such receptors are known to the art and are reviewed by de Haas et al. (1995, J Lab Clin Med 126 p330-341). FcγRIIIb is also known as CD16.

The inventors have found that agents that inhibit FcγRIIIb receptor activity, or activation, or expression, according to the first or second aspects of the invention are of great benefit in the treatment of inflammatory conditions. They have established that such agents are able to prevent or reduce the release from granulocytes of inflammatory mediators into tissue but, surprisingly have no, or minimal, effect on granulocyte mediated killing of phagocytosed pathogens.

Although the inventors do not wish to be bound by any hypothesis, they believe that the advantageous properties of such agents may be explained by the knowledge of granulocyte function they have gained by performing experiments investigating the activity of FcγRIIIb receptors on granulocytes from inflamed tissues. The major factors that activate infiltrating neutrophils within inflamed tissues (e.g. rheumatoid synovial fluid) are IgG-containing immune complexes. The inventors believe such complexes produce activation via different processes. Soluble complexes do not activate unprimed neutrophils, but rather stimulate a rapid and extensive secretion of ROIs and granule enzymes from cells previously primed with agents such as cytokines, particularly TNFα, IL-1β and GM-CSF which are present in high concentration in RA. Such secretion has the potential to cause much damage to neighbouring cells and tissues. Insoluble complexes, on the other hand, activate unprimed as well as primed neutrophils, most of the oxidants are generated intracellularly and there is considerably less capacity to cause tissue damage. These immune complexes appear to activate neutrophils via interaction with their Fcγ receptors and the inventors decided to investigate the roles played by FcγRIIIb in phagocytosis and killing, and in activation by IgG-containing immune complexes. The inventors found impairment of FcγRIIIb receptors resulted in a major decrease in the ability of neutrophils to secrete reactive oxidants in response to soluble immune complexes (as occurs in rheumatoid joints and other inflamed tissues). Furthermore, to their surprise, they found that there is minimal, if any, effect in the ability of granulocytes to phagocytose and kill serum-opsonised Staphylococcus aureus. This observation lead the inventors to realise that selective inhibition of granulocyte function may be practical therapeutically in inflammatory conditions by using agents according to the present invention.

The use of agents according to the first aspect of the invention allows for targeting of neutrophil function, specifically the properties and action of the FcγRIIIb receptor, in a way that surprisingly allows inhibition of the inflammatory process without risk of compromising the immune system.

Genetic data suggests that the absence of the FcγRIIIb receptor on neutrophils does not result in any adverse affects on people. This is based on the observation that, by chance, some individuals screened for the presence of this molecule were found to not possess it. Despite this, they live normal lives and, importantly, are not susceptible to any greater incidence of infection. The only reason they were found not to have this molecule was that they were specifically tested for it. This data suggests that, in contrast to all other targets investigated or used therapeutically for RA, inhibition of this target is extremely safe. Accordingly agents used according to the present invention do not cause side effects commonly seen in prior art biologic drugs.

Whilst agents used according to the first aspect of the invention have significant therapeutic benefit for rheumatoid arthritis, it will be appreciated that such agents also benefit patients suffering from other inflammatory conditions, where neutrophils play an important pathological part. Such conditions include, but are not limited to: Behcet's disease, ANCA-associated vasculitis, systemic vasculitis, cystic fibrosis, asthma, acute gout, chronic obstructive pulmonary disease and Crohn's Disease. As with rheumatoid arthritis, current therapies in these conditions are poor, with limited efficacy and considerable side-effects (including immunosuppression).

Several classes of agent may be used according to the invention to inhibit FcγRIIIb receptor activity. These agents include:
(i) Agents which attenuate transmission at FcγRIIIb receptors (e.g. FcγRIIIb receptor antagonists and partial agonists; and molecules which attenuate FcγRIIIb receptor-effector coupling). These agents may mimic epitopes on Fc regions of IgG, or may interact with FcγRIIIb, thereby preventing its interaction with immune complexes. Such agents may be small peptides based on the known sequences of the Fc region of IgG or the known sequence of FcγRIIIb, for example peptides or modified peptides, or pharmaceutically acceptable salts thereof containing sequences such as -Leu-Pro-Asp-Ala-Arg-His-Ser-Thr-Thr-Gln-Pro-Arg-Lys- (SEQ ID No 1). The sequence may be A-Leu-Pro-Asp-Ala-Arg-His-Ser-Thr-Thr-Gln-Pro-Arg-Lys-B where A and B are any amino acids. Alternatively they may be chemical entities whose structure is such that they interfere with IgG and FcγRIIIb interaction. Alternatively the agents may interfere with the signalling pathways that link FcγRIIIb ligation and activation of neutrophil secretory processes. Such signalling molecules will include, but are not restricted to src-like tyrosine kinases, MAP kinases (including JNK, p38 MAP kinases and ERK), protein kinase C and isotypes thereof.
(ii) Agents that increase shedding of FcγRIIIb from its GPI anchor (e.g. PI-PLC, proteases and activators thereof).

(iii) Agents which reduce FcγRIIIb receptor expression and/or transcription. (e.g. anti-sense molecules or si (small interfering) RNA molecules for the FcγRIIIb receptor gene). These antisense molecules may be based on sequences within the FcγRIIIb gene, that are specific to this gene, within the sequence:

| | |
|---|---|
| TCTTTGGTGA CTTGTCCACT CCAGTGTGGC ATCATGTGGC AGCTGCTCCT | (SEQ ID No. 2) |
| CCCAACTGCT CTGCTACTTC TAGTTTCAGC TGGCATGCGG ACTGAAGATC | |
| TCCCAAAGGC TGTGGTGTTC CTGGAGCCTC AATGGTACAG CGTGCTTGAG | |
| AAGGACAGTG TGACTCTGAA GTGCCAGGGA GCCTACTCCC CTGAGGACAA | |
| TTCCACACAG TGGTTTCACA GTGAGAGCCT CATCTCAAGC CAGGCCTCGA | |
| GCTACTTCAT TGACGCTGCC ACAGTCAACG ACAGTGGAGA GTACAGGTGC | |
| CAGACAAACC TCTCCACCCT CAGTGACCCG GTGCAGCTAG AAGTCCATAT | |
| CGGCTGGCTG TTGCTCCAGG CCCCTCGGTG GGTGTTCAAG GAGGAAGACC | |
| CTATTCACCT GAGGTGTCAC AGCTGGAAGA ACACTGCTCT GCATAAGGTC | |
| ACATATTTAC AGAATGGCAA AGACAGGAAG TATTTTCATC ATAATTCTGA | |
| CTTCCACATT CCAAAAGCCA CACTCAAAGA TAGCGGCTCC TACTTCTGCA | |
| GGGGGCTTGT TGGGAGTAAA AATGTGTCTT CAGAGACTGT GAACATCACC | |
| ATCACTCAAG GTTTGGCAGT GTCAACCATC TCATCATTCT CTCCACCTGG | |
| GTACCAAGTC TCTTTCTGCT TGGTGATGGT ACTCCTTTTT GCAGTGGACA | |
| CAGGACTATA TTTCTCTGTG AAGACAAACA TTTGAAGCTC AACAAGAGAC | |
| TGGAAGGACC ATAAACTTAA ATGGAGAAAG GACCCTCAAG ACAAATGACC | |
| CCCATCCCAT GGGAGTAATA AGAGCAGTGG CAGCAGCATC TCTGAACATT | |
| TCTCTGGATT TGCAACCCCA TCATCCTCAG GCCTCTC | |

siDNA represents a preferred agent for use according to the invention. When the agent is siDNA the forward oligonucleotide (i.e. the sense siRNA—which may be a sequence of between 15 and 23 nucleotides) may be linked to the reverse complementary antisense siRNA sequence by a spacer. For instance, 5-6 Ts may be added to the 3′ end of the oligonucleotides. The reverse siDNA oligonucleotide preferably has a 4-nucleotide overhang, which generates suitable restriction sites, added to the 5′ and 3′ end of the sequence. This allows for subsequent cloning. The RNA transcript derived from such oligonucleotides is likely to fold back and form a stem-loop structure comprising and loop with 2-3 Uracil bases at the 3′ end. Such RNA transcripts can be annealed and ligated into suitable vectors for expression in human myeloid stem cells. As disruption of FcγRIIIb but not FcγRIIIa expression is required, oligonucleotides may be designed that target FcγRIIIb mRNA beyond the SER203 codon.
(iv) Agents may also block transcription of the FcγRIIIb gene, for example by interfering with transcription factors regulating its expression or by binding to regions of DNA in the promoter/regulatory region of the FcγRIIIb gene to prevent binding of transcription factors. Alternatively, agents may result in deletion of part or all of the FcγRIIIb gene such that transcription of the gene is prevented or the transcript generated encodes a non-functional protein.
(v) Agents that can block expression of, or delete the FcγRIIIb gene in human adult myeloid stem cells such that re-infusion of genetically modified myeloid stem cells can be used to treat patients with inflammatory conditions by stem cell gene therapy.
(vi) Agents which inhibit synthesis or release of endogenous FcγRIIIb agonists (e.g immune-complexes) by decreasing the synthesis of precursors or decreasing the conversion of precursors into FcγRIIIb receptor-activating ligands.
(vii) Agents which increase the rate of inactivation or metabolism of FcγRIIIb receptor agonists.

The agent may modulate any type of Fcγ receptor provided that FcγRIIIb receptor activity is inhibited. However it is preferred that the agent selectively inhibits the activity of FcγRIIIb receptors.

By “selectively” we mean the agent inhibits FcγRIIIb receptor activity or activation to a greater extent or at lower doses than other types of Fcγ receptor.

Agents that attenuate transmission at FcγRIIIb receptors ((i) above) represent preferred agents for use according to the invention. Receptors may be blocked with small molecules or peptide based antagonists. Alternatively neutralising antibodies (or active fragments thereof) may be raised against FcγRIIIb receptors (using conventional antibody generating methods). Such antibodies (or active fragments thereof) can be used to bind to cell surface FcγRIIIb and thereby reduce receptor activity. Instead of whole antibodies, F(ab′)₂ or Fab fragments may be used.

A most preferred example of an antibody that may be used according to the invention is the monoclonal antibody 3G8 (available from Medarex, Inc., Annandale, N.J., USA) and F(ab′)₂ or Fab fragments thereof. An alternative antibody is CB16.

Agents that increase receptor shedding ((ii) above) may be used in conjunction with other molecules that promote mobilisation of FcγRIIIb receptors to the cell surface and thereby promote depletion of the receptor from the granulocyte. For instance, PI-PLC (or mimics thereof) may be used in conjunction with TNFα (or mimics thereof). Such agents may be phosphatidylinositol-specific phospholipase C, proteases or agents that perform the same function. Agents that activate these phospholipases or proteases may be used.

The agents (and compositions or medicaments containing them) may be used to treat many types of inflammatory condition. Preferably the agents are used to treat Behcet's disease, ANCA associated vasculitis, systemic vasculitis, gout, cystic fibrosis, asthma, Chronic Obstructive Pulmonary Disease or Crohn's Disease. Most preferably the agents are used to treat Rheumatoid Arthritis.

The agents may also be used in combination with other therapies for treating inflammatory conditions and may also be used when prophylactic treatment is considered medically necessary.

The agents are preferably used to treat human subjects but may also be used for veterinary purposes.

The agents may be formulated in a number of ways depending, in particular on the manner in which the composition/medicament is to be used. Thus, for example, the agent may be formulated in the form of a powder, tablet, capsule, liquid, ointment, cream, gel, hydrogel, aerosol, spray, micelle, liposome or any other suitable form that may be administered to a person or animal. It will be appreciated that the vehicle for the agent should be one that is well tolerated by the subject to whom it is given and enables delivery of the compound to the inflamed tissue.

The agent (e.g. an antibody or antibody fragment) may be provided as a powder. Such a powder may be dissolved in diluent to form a stock solution. A suitable diluent is a sterile saline solution or other suitable aqueos diluent. The diluent may also contain a bactericidal agent to ensure sterility. This stock solution can be administered directly (e.g. subcutaneously, intramuscularly, intradermally, intra-articular or intravenously) or be diluted further in a physiologically neutral solution, such as normal saline, for intravenous infusion.

In another preferred format, the agent is provided as a protein or peptide as powder, which may also be dissolved in diluent to form a stock solution. A suitable diluent is sterile saline solution, or other suitable diluent. This may also contain a bactericidal agent to ensure sterility. This stock solution might be administered directly: subcutaneously, intramuscularly, intradermally, intra-articular, intravenously, or be diluted further in a physiologically neutral solution, such as normal saline, for intravenous infusion.

In another preferred format an antisense molecule is provided in an aqueous buffer.

In another preferred format the agent is provided in a capsule format. This will consist of an outer gelatine coat containing active agent in the range of 10 μg to 1 g, together with appropriate stabilising agent and packing agents.

In another preferred format the agent is provided in a tablet format. This may comprise talc, sucrose and the active agent in pharmaceutically suitable proportions.

When used in a combination therapy, the agent may be formulated in a single composition which also comprises other therapeutically active compound(s). Alternatively the agent and other therapeutically active compound may be formulated in separate formulations and co-administered to the subject either simultaneously or sequentially.

The agents may be used in a number of ways. For instance, systemic administration may be required in which case the agent may be contained within a composition which may, for example, be ingested orally in the form of a tablet, capsule or liquid. Alternatively the agent may be administered by injection into the blood stream. Injections may be intravenous (bolus or infusion) or subcutaneous (bolus or infusion), intra-articular or intramuscular. Administration may also be by inhalation or intranasally.

The agent may also be incorporated within a slow or delayed release device. Such devices may, for example, be inserted under the skin and the agent may be released over weeks or even months. On the other hand, transdermal delivery might be used to achieve the same end. Such devices may be particularly useful for patients requiring long term and/or continuous therapy for an inflammatory condition. The devices may be particularly advantageous when an agent is used which would normally require frequent administration (e.g. at least daily ingestion of a tablet or daily injection).

When the agent is an antibody, antibody fragment, peptide or protein it is preferred that the agent is administered parenterally, by subcutaneous or intramuscular injection or intravenous infusion. Subcutaneous injection may be as a bolus injection performed at regular intervals from once a day to once a month. Alternatively, this may be delivered constantly by continuous subcutaneous infusion by a suitable delivery pump or device. Intramuscular injection should be between once a week to once every two months. Intravenous infusion should be either as bolus, over a few minutes or infusion over a period of hours, repeated at intervals ranging from once a week to once every two months.

When the agent is to be administered intra-articularly, it should be diluted appropriately and injected directly into an inflamed joint under aseptic conditions. Injections shall be repeated at daily intervals until the inflammation has resolved. The course of injections shall be repeated if further inflammation occurs.

When the agent is to be orally ingested in the form of a tablet, capsule or liquid, it should be taken up to four times per day, before food.

It will be appreciated that the agents may be administered by other conventional routes (e.g. intranasally, transdermally, or by enema) using methods known to the art.

It will be appreciated that the amount of an agent required is determined by biological activity and bioavailability which in turn depends on the mode of administration, the physicochemical properties of the agent employed and whether or not it is to be used in combination therapy.

The frequency of administration will also be influenced by the abovementioned factors and particularly the half-life of the agent within the subject being treated.

Known procedures, such as those conventionally employed by the pharmaceutical industry (e.g. in vivo experimentation, clinical trials etc), may be used to establish specific formulations of agents and precise therapeutic regimes (such as daily doses of the compounds and the frequency of administration).

Generally, a daily dose of between 0.1 μg/kg of body weight and 1.0 g/kg of body weight of an agent which inhibits FcγRIIIb receptor activity may be used for the treatment of the inflammatory condition depending upon which specific agent is used. More preferably the daily dose is between 0.01 mg/kg of body weight and 100 mg/kg of body weight and most preferably 0.05-10 mg/kg of body weight.

Purely by way of example a suitable dose of 3G8 antibody against the FcγRIIIb receptor is between 0.01 mgs/kg/day and 100 mgs/kg/day (depending upon the health status of the individual). It is preferred that a single dose of between about 5 and 50 mgs, more preferably of between about 10 and 25 mgs of such an antibody is given as a single dose to treat an inflammatory condition (e.g. RA). We have found that between 2 and 20,000 ng of 3G8 is sufficient to inhibit the FcγRIIIb receptors of 0.5×10⁶ neutrophils and thereby reduce an inflammatory reaction. Most preferably about 200 ng of 3G8 antibody is used to inhibit the FcγRIIIb receptors of 0.5×10⁶ neutrophils. It will be appreciated that the exact amount used will depend upon the individual subject and the type of inflammatory condition being treated.

By way of further example, when PI-PLC (or a similar enzyme is used) it is preferred that the concentration in the tissue being treated is at least 0.1 U/ml and more preferably at least 0.4 U/ml. For instance, when PI-PLC is used to treat RA, it is preferred that the synovial fluid of an arthritic joint contains at least 0.12 U/ml and most preferably about 0.96 U/ml.

Daily doses may be given as a single administration (e.g. a daily tablet for oral consumption or as a single daily injection). Alternatively the agent used may require administration twice or more times during a day. Alternatively a slow release device may be used to provide optimal doses to a patient without the need to administer repeated doses.

A preferred means of using protein or peptide agents which inhibit FcγRIIIb receptor activity for the treatment of an inflammatory is to deliver the protein or peptide to the site of inflammation by means of gene therapy. For instance, gene therapy may be used to decrease expression of FcγRIIIb receptors, increase expression of enzyme(s) responsible for the degradation of endogenous FcγRIIIb receptor agonists, increase expression of a protein which promotes breakdown or desensitisation of FcγRIIIb receptors (e.g. PI-PLC), increase expression of a protein which promotes breakdown of FcγRIIIb receptor agonists or for the purposes of expressing a peptide inhibitor of FcγRIIIb receptors. Therefore according to a third aspect of the present invention there is provided a delivery system for use in a gene therapy technique, said delivery system comprising a DNA molecule encoding for a protein which directly or indirectly inhibits FcγRIIIb receptor activity, said DNA molecule being capable of being transcribed to allow the expression of said protein and thereby treat an inflammatory condition.

The delivery systems according to the third aspect of the invention are highly suitable for achieving sustained levels of a protein or peptide which directly or indirectly inhibits FcγRIIIb receptor activity over a longer period of time than is possible for most conventional therapeutic regimes. The delivery system may be used to induce continuous protein or peptide expression from cells in the inflamed tissue (e.g. an arthritic joint) that have been transformed with the DNA molecule. Therefore, even if the protein has a very short half-life as an agent in vivo, therapeutically effective amounts of the protein may be continuously expressed from the treated tissue.

Furthermore, the delivery system of the invention may be used to provide the DNA molecule (and thereby the protein or peptide which is an active therapeutic agent) without the need to use conventional pharmaceutical vehicles such as those required in tablets, capsules or liquids.

The delivery system of the present invention is such that the DNA molecule is capable of being expressed (when the delivery system is administered to a subject) to produce a protein or peptide that directly or indirectly has activity for inhibiting FcγRIIIb receptor activity. By “directly” we mean that the product of gene expression per se has the required activity (e.g. a protein with receptor neutralising activity). By “indirectly” we mean that the product of gene expression undergoes or mediates (e.g. as an enzyme) at least one further reaction to provide an agent effective for inhibiting FcγRIIIb receptor activity and thereby treating the inflammatory condition.

The DNA molecule may be contained within a suitable vector to form a recombinant vector. The vector may for example be a plasmid, cosmid or phage. Such recombinant vectors are highly useful in the delivery systems of the invention for transforming cells with the DNA molecule.

Recombinant vectors may also include other functional elements. For instance, recombinant vectors can be designed such that the vector will autonomously replicate in the cell. In this case, elements which induce DNA replication may be required in the recombinant vector. Alternatively the recombinant vector may be designed such that the vector and recombinant DNA molecule integrates into the genome of a cell. In this case DNA sequences which favour targeted integration (e.g. by homologous recombination) are desirable. Recombinant vectors may also have DNA coding for genes that may be used as selectable markers in the cloning process.

The recombinant vector may also further comprise a promoter or regulator to control expression of the gene as required.

The DNA molecule may (but not necessarily) be one that becomes incorporated in the DNA of cells of the subject being treated. Undifferentiated cells may be stably transformed leading to the production of genetically modified daughter cells (in which case regulation of expression in the subject may be required e.g. with specific transcription factors or gene activators). Alternatively, the delivery system may be designed to favour unstable or transient transformation of differentiated cells in the subject being treated. When this is the case, regulation of expression may be less important because expression of the DNA molecule will stop when the transformed cells die or stop expressing the protein (ideally when the inflammatory condition has been treated or prevented).

The delivery system may provide the DNA molecule to the subject without it being incorporated in a vector. For instance, the DNA molecule may be incorporated within a liposome or virus particle. Alternatively the “naked” DNA molecule may be inserted into a subject's cells by a suitable means e.g. direct endocytotic uptake.

The DNA molecule may be transferred to the cells of a subject to be treated by transfection, infection, microinjection, cell fusion, protoplast fusion or ballistic bombardment. For example, transfer may be by ballistic transfection with coated gold particles, liposomes containing the DNA molecule, viral vectors (e.g. adenovirus) and means of providing direct DNA uptake (e.g. endocytosis) by application of the DNA molecule directly to the inflamed tissue topically or by injection.

The inventors work on FcγRIIIb receptors has enabled them to develop a screening method to test whether or not compounds will have efficacy for treating inflammatory conditions according to the invention. Therefore according to a fourth aspect of the invention there is provided a screening method for identifying anti-inflammatory agents comprising the steps of contacting a compound with FcγRIIIb receptors and measuring inhibition of receptor activity or activation by said compounds.

According to a fifth aspect of the present invention there is provided an anti-inflammatory agent identified by the screening method according to the fourth aspect of the invention.

The screening method according to the fourth aspect of the invention is useful for screening compound libraries to evaluate whether or not candidate compounds are able to inhibit FcγRIIIb receptor activity or activation. Any compounds that cause such inhibition may be subjected to detailed study to assess whether or not they will be useful for treating the inflammatory conditions discussed above.

Inhibition of FcγRIIIb receptor activity or activation may be assessed in a number of ways.

The screening methods according to the method of the fourth aspect of the invention may be conducted on receptors on the cell surface of isolated neutrophils. Alternatively a whole blood sample may be used.

The screening methods are also preferably carried out on samples containing, or derived from, primed neutrophils. Neutrophils may be primed with a number of agents including TNFα, IL-1β, IL-6, IL-8, IFNγ, lipopolysaccharide (LPS), granulocyte macrophage colony-stimulating factor (GM-CSF) and granulocyte colony-stimulating factor (G-CSF), the chemotactic peptide, fMet-Leu-Phe, cytochalasin B,.LTB₄, C5a.

The screening method according to the fourth aspect of the invention may be centred around a receptor binding assay. When this is the case, compounds that are candidate ligands may be labelled to allow binding to be monitored. By way of example only, the binding assay may involve the preparation of cell membranes comprising the FcγRIIIb receptor; such membranes may be incubated with a known amount of a radiolabelled ligand; the membranes harvested (e.g using a cell harvester and capturing the membranes on filter papers); and then the amount of radiolabelled ligand bound to the membranes quantified by scintillation counting. Alternatively the ligand may be fluorescently labelled, and fluorescence measurements used to monitor binding. Receptor:ligand binding assays may alternatively be based on biosensor-based assays in which the receptor (either purified or recombinant) or membranes or lioposomes containing the receptor are bound to a chip and incubated with ligand. The effects of chemical entities to interfere with binding may then be tested. Binding assays may also be based on FRET (fluorescence resonance energy transfer) technologies. An example of this screening assay is that the ligand is labelled with one type of fluorescent probe whilst the receptor is labelled with a different fluorescent probe. When binding of ligand to receptor occurs, then FRET is measured. Molecules which prevent this FRET potentially block receptor: ligand interactions.

A preferred method for assessing whether or not candidate compounds will inhibit FcγRIIIb receptors, involves the use of Fab/F(ab′)₂ fragments of anti-CD32 (FcγRII) and anti-CD16 (FcγRIIIb) monoclonal antibodies. This method involves the following steps:

1st Round Screen

(1) Coat plates with saturating concentrations of anti-CD16 Fab/F(ab′)₂ fragments;

(2) isolate neutrophils (preferrably under conditions that minimise cellular priming)

(3) Prime neutrophils (e.g. by incubation with TNFα or other priming agents);

(4) Add candidate compounds (in buffer) to each well of the plate;

(5) Add primed neutrophils to each well, together with luminol; and

(6) The chemiluminescence generated is measured as an indication of the receptor mediated signalling pathway which terminates in the production of reactive oxygen metabolites. Reactive oxygen metabolites that contribute to chemiluminescence include superoxide anions, hydrogen peroxide, HOCl, singlet oxygen, hydroxyl radicals, NO and oxidants formed as a result of reactions involving these molecules. The oxidants either oxidise luminol or generate an excited state. As the electrons in “activated” luminol return to their ground state, photons are emitted, detected as light emission.

Compounds of interest will inhibit neutrophil chemiluminescence stimulated by ligation of FcγRIIIb and consequent reduction of the subsequent respiratory burst.

2nd Round Screen (Optional)

(7) Coat microtitre plates with saturating concentrations of anti-CD32 Fab/F(ab′)₂ fragments;

(8) Add compounds selected from 1^{st} Round Screen to wells;

(9) Add primed neutrophils to each well together with luminol;

(10) Measure chemiluminescence.

Compounds that inhibit this 2^{nd} round activity do NOT have the required selectivity. Therefore candidate compounds that inhibit neutrophil activity in the first screen, but not the second screen, are potential selective inhibitors of FcγRIIIb receptors and may be useful as anti-inflammatory agents.

A preferred protocol for performing these 1^{st} and 2^{nd} screens is given in Example 2.

Another preferred method is described in Example 3. This screening method is similar to the preferred method described above except that it allows the measurement of receptor activity from whole blood rather than isolated neutrophils.

Instead of a binding assay (such as described above), it will be appreciated that the method of the fourth aspect of the invention may be based on a functional assay. Such an assay may be performed to monitor a physiological consequence of receptor activation (e.g second messenger production). Such assays may be based upon the release of a granule enzyme from primed neutrophils, after ligation of FcγRIIIb and/or FcγRII. The released granules enzyme to be measured would include but is not restricted to myeloperoxidase, elastase (or other proteases), defensins, permeability-inducing factors, gelatinase or gelatinase-binding protein, acid hydrolases.

Activation of the production of an intracellular signalling molecule may also be used as the output response of an assay to measure receptor activation. This would include but is not restricted to measurements of intracellular Calcium ions. Such assays for intracellular Calcium may be based on fluorescence measurements of intracellular calcium indicators. Activation of neutrophils could also be achieved by fluorescence based assays that detect activation of the respiratory burst following receptor activation. Such assays could involve the use of fluorescent indicators (e.g. 1,2,3 dihydrorhodamine) that undergo a change in their fluorescent properties when they react with oxidants generated by activated neutrophils during the respiratory burst.

As an alternative to activating FcγRIIIb and FcγRII function by use of antibodies or antibody fragments, soluble and insoluble IgG-containing immune complexes may be used to initiate the cellular response.

The present invention will now be further illustrated, by way of example, with reference to the accompanying drawing, in which:

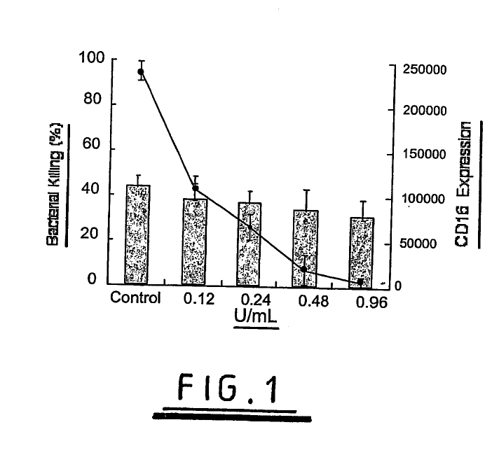

FIG. 1 illustrates enzymic removal of FcγRIIIb from neutrophils and effects on bacterial killing as discussed at 1.2.1 of Example 1;

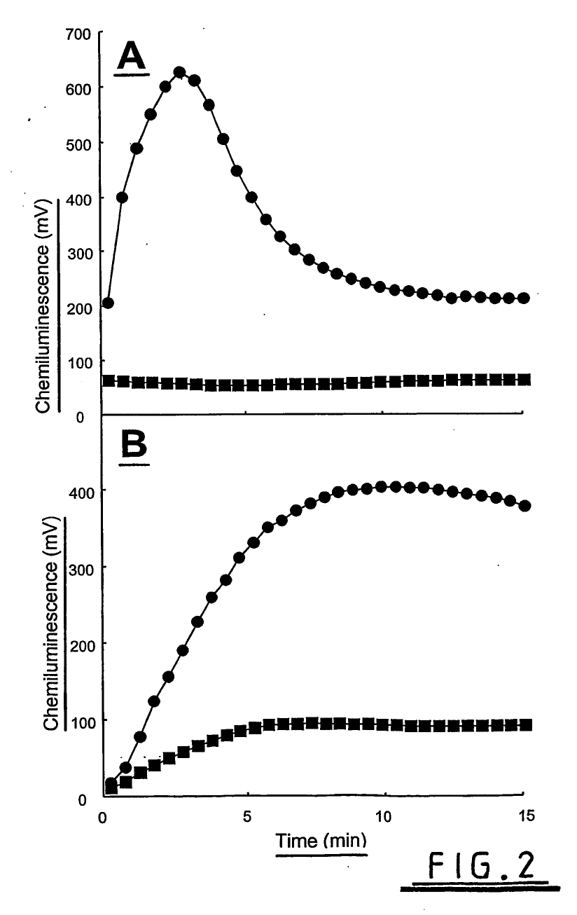

FIG. 2 illustrates the effect of FcγRIIIb expression on activation by IgG-containing immune complexes (either soluble (A) or insoluble (B)) as discussed at 1.2.2 of Example 1;

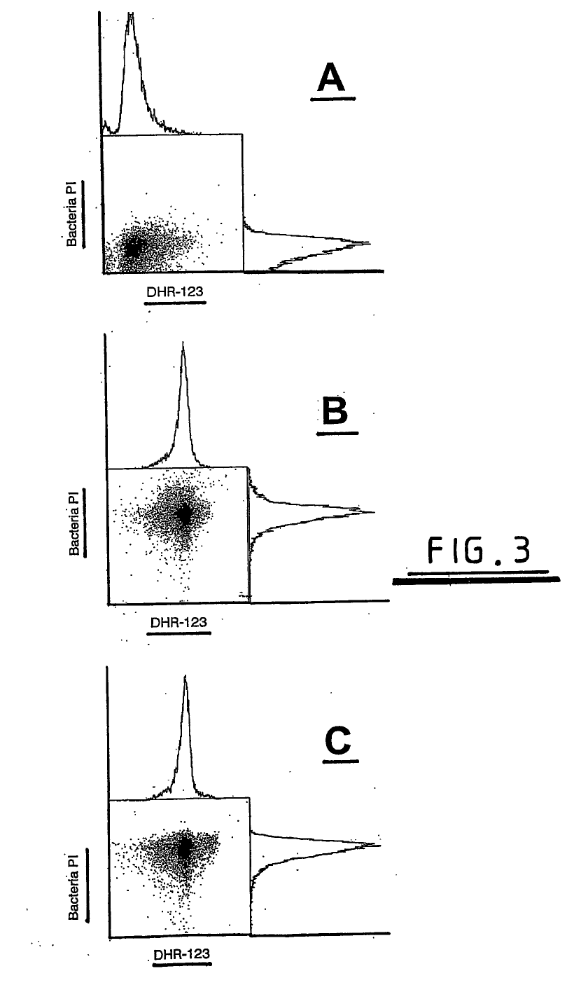

FIG. 3 illustrates phagocytosis of heat killed S. aureus and respiratory burst activation as discussed at 1.2.4 of Example 1;

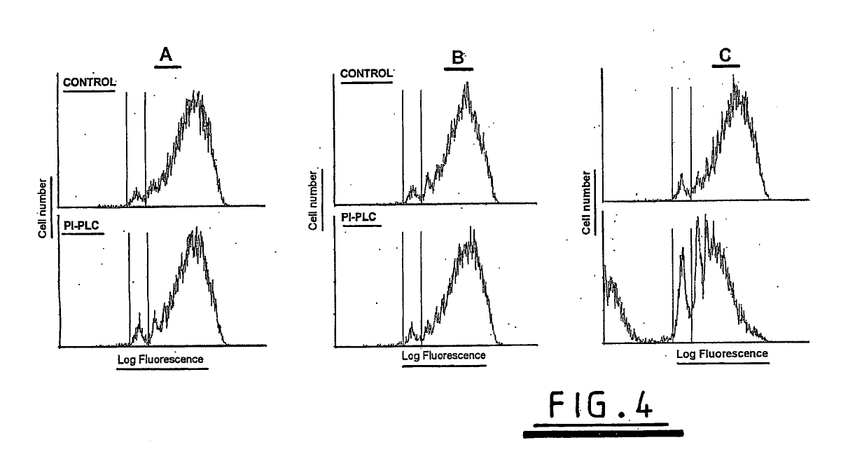

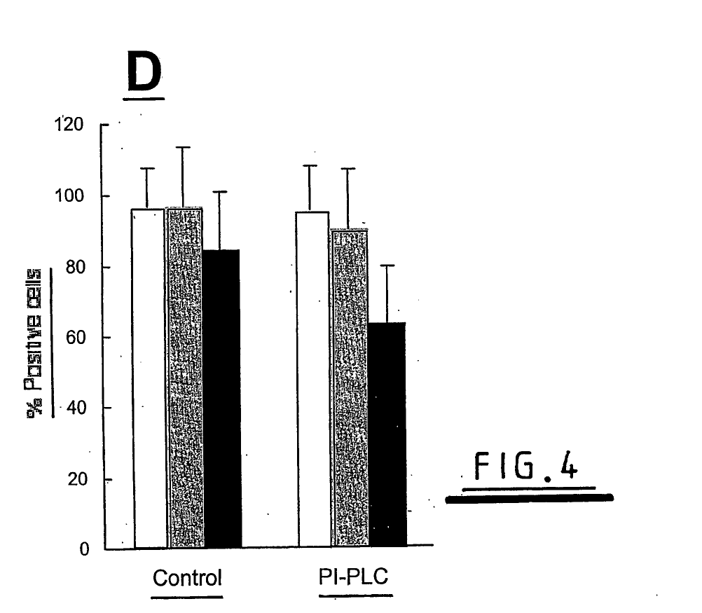

FIG. 4 illustrates phagocytosis of latex particles as discussed at 1.2.5 of Example 1;

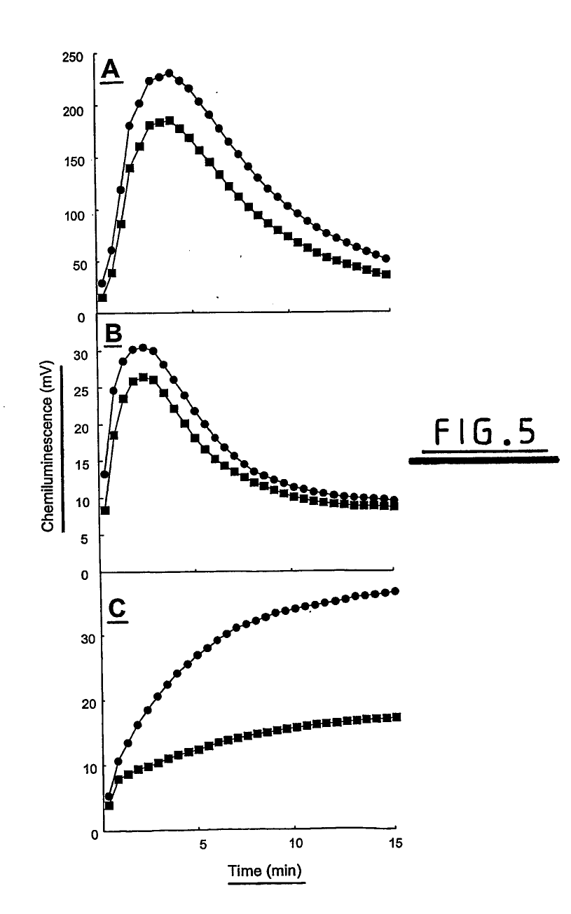

FIG. 5 illustrates respiratory burst activation during phagocytosis of latex particles as discussed at 1.2.6 of Example 1; and

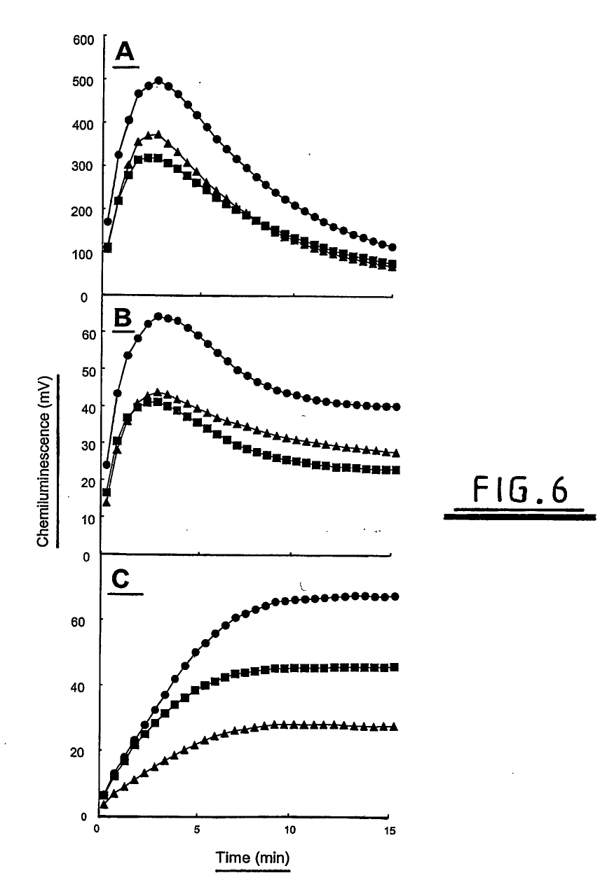

FIG. 6 illustrates the effect of blocking FcγRIIIb and FcγRII on uptake of latex particles as discussed at 1.2.6 of Example 1.

### EXAMPLE 1

Experiments were performed to illustrate that agents which inhibit FcγRIIIb receptors may be used to treat inflammatory conditions without impairing the ability of granulocytes to kill pathogens.

1.1. Materials and Methods

1.1.1 Materials.

Luminol, latex particles (1 μm diameter), FITC-latex particles (1 μm diameter), rabbit anti-human albumin, human IgG, human albumin, propidium iodide, dihydrorhodamine-123, Quantum Simply Cellular Microbeads, and TNF-α were from Sigma (Poole, Dorset, UK). PI-PLC (phosphatidylinositol-phospholipase C) was from ICN Biochemicals (Costa Mesa, Calif.), FITC-labelled Leul 1b (anti-CD16) was from Becton Dickinson (Franklin Lakes, N.J., USA) and RPMI 1640 was from GibcoBRL (Life Sciences, Paisley, UK). Neutrophil Isolation Medium (NIM) was from Cardinal Associates, (Sante Fe, N. Mex., USA). Monoclonal antibodies (IV.3 and 3G8), and F(ab′)₂ fragments of monoclonal antibody 3G8 (recognising FcγRIIIb) and Fab fragments of IV.3 (recognising FcγRII) were from Medarex, Inc (Annandale, N.J.). All other reagents were of the highest purity available.

1.1.2 Preparation of Neutrophils.

The study was approved by the Liverpool Research Ethics Committee. Neutrophils were separated from heparinised human blood from healthy volunteers using a one-step centrifugation procedure on NIM, as previously described (Edwards (1996) Methods 9 p563-577). After hypotonic lysis of erythrocytes, they were suspended in RPMI 1640 medium and counted using a Fuchs-Rosenthal haemocytometer slide. Cell purity (Wright's staining) and viability (trypan blue exclusion) were routinely >97% and >98% immediately after isolation.

1.1.3 Neutrophil Incubations.

Neutrophils were incubated at 5×10⁶ cells/ml in RPMI 1640 medium containing 25 ng/ml TNFα for 15 min at 37° C., with gentle agitation. Primed neutrophils were incubated in 2×10⁶ cells in the absence of (control) or presence of PI-PLC (over the range 0.12-0.96 U/ml) for 30 min at 37° C. with gentle agitation to remove surface FcγRIIIb.

1.1.4 Flow Cytometry.

After incubations as described above, neutrophils were resuspended in phosphate buffered saline (PBS: 10 mM potassium phosphate, 0.9 % NcCl, pH 7.4) containing 3 mM sodium azide and incubated with saturating amounts of FITC-labelled Leu11b at 4° C. for 30 min. After this, cells were washed and fixed with 1% paraformaldehyde in PBS and stored in the dark at 4° C. until analysis using an Ortho Diagnostics Cytron flow cytometer. A total of 10,000 gated events were analysed. Fluorescence values were then quantified for antibody binding sites per cell (ABC) using a Quantum Simply Microbeads kit, according to the manufacturer's instructions

1.1.5 Immune Complex Preparation.

Synthetic immune complexes were made from human serum albumin (HSA) and rabbit anti-HSA antibodies as previously described (Edwards et al. (1997) Ann N Y Acad Sci 832 p341-357; Watson & Edwards (1998) Biochem Biophys Res Commun 247 p819-826; & Watson et al. (1997) J Biol Chem 272 p1 7944-17951). The antigen was titrated against constant antibody concentration and the A45o was measured to identify equivalence. The soluble complexes were formed at 6 x antigen equivalence and were briefly centrifuged (2 min at 13000 g in a microfuge) to remove any contaminating insoluble immune complexes that may have been present. Soluble complexes were formed at 180 μg/ml antigen and 125 μg/ml of antibody. A 10% (v/v) solution of complexes was used routinely for neutrophil stimulation.

1.1.6 Growth, Opsonisation and Killing of Staphylococcus aureus.

S aureus (Oxford) was grown overnight on nutrient agar plates at 37° C. The cells were then washed off the plates and suspended in sterile PBS. The number of cells was determined by A₅₅₀ measurements and suitable calibration curves. Opsonisation using pooled human serum from healthy donors (stored in aliquots at −20° C.) was achieved by incubating bacteria (5×10⁸/ml) with a 10% heat-inactivated human serum (v/v), final conc.) for 30 min at 37° C. (59,60) prior to the addition of 3 vol. cold sterile PBS and whirlimixing for 30 s. After this the opsonised bacteria were centrifuged at 1000 g for 15 min, washed 3 times in sterile PBS and finally suspended at known concentrations. Opsonised bacteria were mixed with neutrophils at a ratio of 10:1 and incubated with gentle agitation for 30 min at 37° C. The neutrophil: bacteria mixture was diluted 2000 fold in water and then vigorously whirlimixed for 1 min to lyse the neutrophils and dilute the bacteria. Aliquots were then plated (by spread plating) onto nutrient agar plates and colonies (maximum of 500 per plate) were counted after overnight incubation. Killing was expressed as a percentage of the decrease in bacterial count in suspensions incubated with neutrophils compared to control suspensions incubated of bacteria.

1.1.7 Phagocytosis of Heat-Killed Bacteria.

S aureus were heat killed by incubation at 60° C. for 30 min, washed twice and then resuspended in PBS containing 30 μM propidium iodide (PI). The suspension was incubated in the dark at 4° C. for 2 h. The fluorescent bacteria were washed 3 times in Hanks Balanced Salts Solution (HBSS) containing 0.1% gelatin and opsonised as described above. They were then incubated with neutrophils at a ratio of 10 bacteria: 1 neutrophil, in the dark for 30 min at 37° C. with gentle agitation. After incubation, the neutrophils were pelletted by centrifugation, washed twice and then suspended in PBS containing 5 mM EDTA, 3 mM sodium azide and 1% paraformaldehyde. Cells were analysed immediately by flow cytometry and red fluorescence was collected through a 620 nm long pass filter. 10,000 gated events were collected in the neutrophil gate. Phagacytosis of PI-stained bacteria was vertified by confocal microscopy.

1.1.8 Opsonisation and Phagocytosis of Latex Beads.

1 μm diameter latex beads were incubated for 2 h with either pooled human serum or with 5 mg/ml human IgC, in HBSS with gentle agitation. The beads were then washed 3 times in RPMI 1640 medium. For flow cytometry, FITC-labelled beads were used and subsequently incubated with neutrophils at a ratio of approx 10 beads to 1 neutrophils. As indicated, some experiments also used unopsonised FITC-latex beads. For chemiluminescence measurements (see below), non FITC-latex beads were used and, as indicated, were opsonised as described above.

1.1.9 Measurement of Reactive Oxidant Production.

Chemiluminescence was measured at 37° C. in neutrophil suspensions (5×10⁵ ml) in RPMI 1640 medium that was supplemented with 10 μM luminsol using an LKB 1251 luminometer. Cells were stimulated by addition of soluble immune complexes or opsonised latex particles. Intracellular H₂0₂ production was also measured by flow cytometry using dihydrorhodamine-123. Neutrophils and PI-stained bacteria were incubated as above, and after 15 min incubation, dihydrorhodamine- 123 was added to a final concentration of 2.5 μM. After a further 15 min incubation at 37° C. (in the dark), suspensions were washed and analysed immediately by flow cytometry: green fluorescence from dihydrorhodamine-123 was collected through a 530 nm band pass filter and red fluorescence from the PI-stained bacteria collected through a 620 nm long pass filter

1.1.10 Locking Fcγ Receptor Function.

0.5×10⁶ neutrophils/ml in RPMI 1640 medium were incubated at 37° C. with 25 ng TNFα and then incubated for a further 15 min with either 200 ng for a Fab fragment of 3G8 (anti- CD16) or 200 ng of a F(ab′)₂ fragment of IV3 (anti-CD32). Luminol was added to a final concentration of 10 μM and then unopsonised, serum-opsonised or IgC-opsonised latex beads were added and chemiluminescence measured.

1.2 Results

1.2.1 Removal of Surface FcγRIIIb.

Neutrophils are known to express high levels of FcγRIIIb on their cell surface but also possess large intracellular pools of this receptor on the membranes of intracellular granules. Freshly-isolated blood neutrophils were therefore incubated for 15 min with 25 ng/ml TNFα in order to mobilise these internal stores of FcγRIIIb to the cell surface. They were then incubated for 30 min with increasing concentrations PI-PLC and then surface FcγRIIIb expression was assessed by flow cytometry.

FIG. 1 shows that this treatment resulted in a decreased surface expression of this receptor in a dose-dependent manner.

For FIG. 1 neutrophils were primed by incubation with 25 ng/ml TNFα for 15 min and then incubated either in the absence (control) or presence of PI-PLC at the concentrations shown for 30 min. Cell surface FcγRIIIb (CD16) was then assayed by flow cytometry with the number of CD16 antibody-binding sites calculated as described in Methods (240,000 per control cell). Values shown (•) are means (±SD, n=20). Primed neutrophils (with and without PI-PLC treatment) were also incubated with serum-opsonised S. aureus for 30 min. Viable bacteria were then enumerated as described in Methods. Values shown ( [symbol] ) are means (±SD, n=10).

At the highest concentrations used, surface FcγRIIIb expression was decreased to <3% of the control, untreated value, as assessed by measuring antibody binding sites per cell: control cells possessed approx, 240,000 antibody binding sites per cell. This treatment did not affect the surface expression of CD32 (FcγRII) or CD11b (data not shown).

1.2.2 Effects of PI-PLC on Activation by IgC-Containing Soluble and Insoluble Immune Complexes.

Primed neutrophils, but not unprimed neutrophils, (data not shown) generated a rapid respiratory burst in response to soluble immune complexes that peaks 2-3 min after addition (FIG. 2A). However, when surface expression of FcγRIIIb was depleted by treatment with PI-PLC as above, the soluble immune complexes failed to activate reactive oxidant. Activation of primed neutrophils by insoluble immune complexes resulted in a much slower stimulation of the respiratory burst and a response decreased by approximately 70% after FcγRIIIb depletion (FIG. 2B).

In FIG. 2, primed neutrophils were pre-incubated in the absence of (•) or presence of 0.96% U/ml PI-PLC (▪) as described in 1.2.1. They were then incubated with either soluble (A) or insoluble (B) immune complexes and luminol chemiluminescence was measured. Values shown are typical of 6 separate experiments.

These data surprised the inventors and suggested to them that soluble and insoluble immune complexes must stimulate neutrophils by distinct activation pathways. These experiments also show that activation of primed neutrophils, of the type found in RA joints, by soluble immune complexes was completely dependent upon surface FcγRIIIb expression, and that this receptor also plays a major role in activation by insoluble immune complexes.

1.2.3 Bacterial Killing by FcγRIIIb Depleted Neutrophils.

It was then necessary to determine if depletion of surface FcγRIIIb had any effect on the ability of neutrophils to phagocytose and kill bacteria. After incubation of control (untreated) neutrophils with (heat-inactivated) serum-opsonised S.aureus, for 30 min at 37° C., only 42.3% of the initial inoculum (±5.3% SEM, n=10) remained viable (FIG. 1). When neutrophils were treated with PI-PLC, bacterial killing was minimally, if at all, affected. For example, at 0.96 U/ml PI-PLC (which resulted in >97% removal of surface FcγRIIIb), bacterial killing was 32.9% (±4.2% SEM, n=10) compared with control values of 45% of the bacteria killed (±5% SEM, n=10, p=0.387). Thus, whilst PI-PLC effectively removed surface FcγRIIIb, no significant inhibition of killing of serum-opsonised S. aureus was observed.

This indicates that killing of serum-opsonised S. aureus is not dependent on surface FcγRIIIb expression and indicated that agents may be used according to the invention without reducing the pathogen killing capabilities of granulocytes.

1.2.4 Effects of PI-PLC on Phagocytosis of Serum Opsonised, Heat Killed Bacteria.

Having shown that killing of serum-opsonised S. aureus was not affected by FcγRIIIb depletion, it was then necessary to determine if binding or phagocytosis of this pathogen were decreased when this receptor was removed. Phagocytosis of serum-opsonised, PI-stained S. aureus was determined by flow cytometry. PI-PLC treatment (that resulted in depletion of over 97% of surface FcγRIIIb) had no effect on phagocytosis compared with control (untreated) values (FIG. 3).

For FIG. 3, S. aureus were killed, stained with proopidium iodide and then opsonised as described in Methods. After 15 min incubation of control (B) or PI-PLC treated (C) neutrophils with these bacteria, 2.5 μM dihydrorhodamine-123 was added and incubation continued for a further 15 min. Two-colour fluorescence distributions were then obtained. (A) shows the lack of phagocytosis and respiratory burst activity when neutrophils were exposed to unopsonised S. aureus. These data represent typical result from at least 10 different experiments.

The ability of neutrophils to generate a respiratory burst during phagocytosis, measured by quantifying dihydrorhodamine-123 fluorescence in the same cells, was similarly unaffected by PI-PLC treatment compared to controls. Confocal microscopy confirmed that almost all of the PI-stained bacteria were taken up into phagocytic vesicles, rather than remaining attached to the cell surface (data not shown). These experiments also demonstrated that unopsonised S. aureus are not phagocytosed by human neutrophils and are incapable of activating a respiratory burst.

1.2.5 Effect of Opsonisation on Phagocytosis of Latex Particles.

The above experiments showed that FcγRIIIb expression played little role in phagocytosis of serum-opsonised S. aureus. The inventors then sought to determine if this receptor was important during phagocytosis of particles opsonised specifically by IgG, rather than the opsonins present in serum. The strain of S. aureus used in these studies was protein A positive and so unsuitable for opsonisation by IgG. Another type of phagocytic particle, namely latex beads, was therefore used, which whilst avidly phagocytosed by neutrophils without opsonisation, can also be opsonised by serum of IgG. Neutrophils avidly phagacytosed unopsonised FITC-latex beads as determined by flow cytometry (FIGS. 4A-C) and confirmed by confocal microscopy (not shown).

In FIG. 4, primed neutrophils were incubated in the absence (control) or presence of 0.96 U/ml PI-PLC as described in 1.2.1 and then incubated with either (A) unopsonised FITC-latex particles or (B) serum opsonised FITC-latex particles or (C) human IgG opsonised FITC-latex particles. Uptake was determined by flow cytometry. The bar in the fluorescence distributions indicates uptake of a single bead per neutrophil. (D) represents cumulative data from 7 different experiments showing uptake of ∠ unopsonised FITC-beads, [symbol] serum-opsonised latex beads and ▪ IgG-opsonised latex beads. Values shown are means (±SD, n=7).

Treatment of neutrophils with PI-PLC had an insignificant effect on uptake of unopsonised particles (Fig.4A) and on uptake of serum opsonised beads (FIG. 4B). However, phagocytosis of IgG-coated beads was markedly decreased by removal of surface FcγRIIIb by PI-PLC (FIG. 4C). In particular, there were increases in the number of neutrophils only phagocytosing a single latex particle and in those that could not phagocytose and particles. Combined results from 7 individual experiments (FIG. 4D) revealed PI-PLC treatment had not significant inhibitory effect on phagocytosis of unopsonised or serum-opsonised beads (p=0.40 and 0.78, respectively, n=7), whilst phagocytosis of IgG-opsonised beads was significantly decreased after PI-PLC treatment(p=0.0022, n=7). These experiments show that surface FcγRIIIb expression plays little role in phagocytosis of unopsonised or serum-opsonised latex particles, but a major role in phagocytosis of IgG-coated particles.

1.2.6 Role of FcγRIIIb Expression in Respiratory Burst Activation During Phagocytosis.

As phagocytosis of IgG-opsonised latex particles was significantly decreased by Fc7RIIIb depletion, shown above, we then sought to determine if activation of the respiratory burst was similarly affected in cells depleted of this receptor. The ability of latex particles to induce a respiratory burst in untreated and FcγRIIIb depleted neutrophils was therefore assessed by chemiluminescence and is illustrated in FIG. 5.

In FIG. 5, neutrophils were pre-incubated as described in 1.2.4 and then incubated with either (A) unopsonised latex particles, (B) serum opsonised latex particles or (C) IgG opsonised latex particles, in the absence of 10 μM luminol. Chemiluminescence was then measured. Values shown are typical of 8 separate experiments. (•) control, untreated cells, (▪) PI-PLC treated cells (0.96 U/ml).

The chemiluminescence profiles of control and PI-PLC treated neutrophils incubated with unopsonised latex particles were extremely similar (FIG. 5A). Likewise, when respiratory burst activity was stimulated by serum-opsonised latex particles, the response of PI-PLC treated cells was not significantly different to controls (FIG. 5B). These results confirm, by an independent assay, that FcγRIIIb plays only a minor role in neutrophil activation in response to these stimuli. The kinetics of activation by IgG-opsonised particles were much slower than those activated by serum-opsonised particles—intending that serum-opsonised particles and IgG-opsonised particles stimulate neutrophils by different signalling/activation pathways. Depletion of surface FcγRIIIb markedly decreased respiratory burst activation only in response to IgG-opsonised particles (Fig. SC), clearly showing that these particles activate neutrophils by pathways critically dependent on FcγRIIIb. Furthermore, these experiments also show that when particles are opsonised with serum, IgG cannot be the major opsonin (a) because the kinetics of activation of serum-opsonised particles and IgG-opsonised particles are so different and (b) because FcγRIIIb depletion has no effect on activation by serum-opsonised particles.

1.2.7 Receptor Blocking Studies.

Finally, as an alternative approach to enzymic depletion of FcγRIIIb, we probed the role of this receptor and FcγRIIIb in receptor blocking studies and the results are illustrated in FIG. 6.

In FIG. 6, neutrophils were pre-incubated without (•) or with Fab/F(ab′)₂ fragments of 3G8 (anti-CD16, ▴) and IV3 (anti-CD32), ▪), as described in Methods. They were then incubated with either (A) unopsonised latex particles, (B) serum opsonised latex particles or (C) IgG opsonised latex particles and luminol chemiluminescence measured. Values shown are typical of 6 separate experiments.

Blocking either FcγRIIIb or FcγRII with F(ab′)₂ /Fab fragments of 3G8 and IV3, respectively, had a slight but equal on activation by unopsonised latex particles (FIG. 6A). Similar results were obtained upon activation by serum-opsonised latex particles (FIG. 6B). However, activation by IgG opsonised latex particles was markedly decreased when FcγRIIIb occupancy was blocked by 3G8 (FIG. 6C). These data again show that FcγRIIIb is important in activation of IgG-opsonised particles, but plays only a minor role in activation by serum-opsonised particles.

1.3 Discussion

The inhibition of pathological neutrophil function to reduce inflammation, without compromising host defence, is an attractive goal for the therapy of inflammatory conditions such as RA, which, until now, was not achievable.

However, in this Example, the inventors illustrate that agents which block signalling through the neutrophil immunoglobulin receptor FcγRIIIb are able to reduce inflammation, without compromising host defence.

FcγRIIIb is tethered on the outer leaflet of the neutophil plasma membrane via a glycosylphosphatidylinositol (GPI) linkage, which may be readily disrupted by treatment with agents such as phosphatidylinositol-phospholipase C (PI-PLC). The inventors therefore used this-agent to remove surface bound receptor and found that it was capable of receptor depletion in a dose dependent manner, without affecting the expression of other cell surface receptors such as FcγRII or CD11b.

Substantial pools of FcγRIIIb are found intracellularly on granule membranes, the inventors therefore also pre-treated neutrophils with TNFα to mobilise these stores to the plasma membrane prior to enzyme treatment. After TNFα treatment approximately 240,000 antibody-binding sites per cell were measured.

The inventors then used a variety of assays to probe the role of FcγRIIIb on neutrophil function. PI-PLC treatment to remove surface FcγRIIIb completely blocked neutrophil secretion in response to soluble immune complexes of the type found in rheumatoid joints (FIG. 2)

However, PI-PLC had no effect on the ability of neutrophils to phagocytose serum-opsonised PI labelled S. aureus, as determined by flow cytometry or confocal microscopy. In parallel with unaltered phagocytosis, respiratory burst activity was also unaffected by PI-PLC mediated surface FcγRIIIb depletion.

Although uptake of unopsonised or serum-opsonised latex (or serum-opsonised S.aureus) was unaffected, uptake and respiratory burst activation of IgG-opsonised latex particles was significantly decreased after PI-PLC treatment. This indicated that FcγRIIIb plays a role in phagocytosis (and presumably killing) of IgG-coated pathogens. Furthermore, blocking FcγRIIIb binding with anti-CD16 F (ab′)₂ fragments also decreased respiratory burst activation during phagocytosis of IgG-opsonised particles. Taken together, these results show clearly that effective phagocytosis and killing of serum-opsonised bacteria (such as S. aureus) does not require expression of functional FcγRIIIb on the cell surface of neutrophils. Presumably other serum proteins (such as complement proteins) can act as efficient opsonins for non- FcγRIIIb dependent phagocytosis and killing. IgG₂, which may be an efficient opsonin in serum, only binds FcγRIIa and is capable of binding to FcγRIIIb. The inventors believe these observations explain why FcγRIIIb gene deficient individuals do not have particular problems in dealing with opportunistic infections by this, or indeed any other, pathogen—a key observation that supports our finding that this molecule does not play a role in host defence against infection but is involved in inflammatory reactions caused by the release of inflammatory mediators into the extracellular environment.

Soluble immune complexes activate a rapid and extensive secretion of reactive oxidants and granule enzymes from primed neutrophils. Not only are such immune complexes found in huge amounts in the synovial fluid of patients with RA, the neutrophils, present in abundance in diseased joints, are primed by the proinflammatory cytokine environment. This phenomenon is of importance in damaging joints in RA. This example illustrates, for the first time, that disruption of FcγRIIIb function completely disrupts activation of secretion of reactive oxidants in response to soluble immune complexes, but has virtually no effect on the ability of human neutrophils to phagocytose and kill serum-opsonised S. aureus. The inventors were therefore motivated to propose the therapeutic blocking of FcγRIIIb ligation or function for the treatment of inflammatory conditions, such as RA, as defined by the present invention.

### EXAMPLE 2

A screening method was developed that may be employed according to the fourth aspect of the invention for screening compounds for their ability to inhibit FcγRIIIb binding or signalling.

2.1. Materials and Methods

2.1.1 Materials & Reagents

Fab/F(ab′)₂ fragments of anti-CD32 (FcγRII) and anti-CD16 (FcγRIIIb) monoclonal antibodies

Hanks Balanced Salts Solution (HBSS)

Luminol

Heparinised human blood

TNFα2.1.2 Method

1 st Round Screen

Coat 384 microtitre plates with saturating concentrations of anti-CD16 Fab/F(ab′)₂ fragments

Isolate neutrophils from human blood under conditions that minimise cellular priming

Prime neutrophils by incubation with TNFα at 10 ng/mL (final conc) for 15 min at 37° C.

Add chemical entity to wells over the concentration range up to 100 μM in a total volume of 5 μL.

Add 10⁴ primed neutrophils to each well, together with HBSS and 10 μM luminol (final conc) in a total volume of 25 μL

Measure chemiluminescence generated for a period of 15 min at 37° C. Compounds of interest will inhibit neutrophil chemiluminescence stimulated by ligation of FcγRIIIb

Negative Controls

Neutrophils added to wells that are not coated with anti-CD16 Fab/F(ab′)₂ Unprimed neutrophils

Positive Controls

Add Diphenylene iodonium (DPI) at a final concentration of 1 μM to wells prior to addition of cells

Select compounds that inhibit neutrophil chemiluminescence for second round of screening.

2^{nd} Round Screen

Coat 384 microtitre plates with saturating concentrations of anti-CD32 Fab/F(ab′)₂ fragments

Add chemical entity selected from first to wells, over the concentration range up to 100 μM in a total volume of 5 μL.

Add 10⁴ primed neutrophils to each well, together with HBSS and 10 μM luminol (final conc) in a total volume of 25 μL

Measure chemiluminescence generated for a period of 15 min at 37° C. Chemical entities that inhibit this activity do NOT have the required selectivity

Chemical entities that inhibit neutrophil activity in the first screen, but not the second screen, are potential selective inhibitors of FcγRIIIb

### EXAMPLE 3

A further screening method was developed that may be employed according to the fourth aspect of the invention for screening compounds for their ability to inhibit FcγRIIIb binding or signalling.

3.1. Materials and Methods

3.1.1 Materials & Reagents—see 2.1.2

3.1.2 Method

1 st Screen

Coat 384 microtitre plates with saturating concentrations of anti-CD16 Fab/F(ab′)₂ fragments

Incubate whole, heparinised blood with TNFα at 50 ng/mL (final conc) for 15 min at 37° C.

Add chemical entity to wells over the concentration range up to 100 μM in a total volume of 5 μL.

Add 1 μL whole blood containing primed neutrophils to each well, together with HBSS and 10 μM luminol (final conc) in a total volume of 25 μL.

Measure chemiluminescence generated for a period of 15 min at 37° C. Compounds of interest will inhibit neutrophil chemiluminescence stimulated by ligation of FcγRIIIb

Negative Controls

Whole blood added to wells that are not coated with anti-CD16 Fab/F(ab′)₂

Positive Controls

Add Diphenylene iodonium (DPI) to wells prior to addition of cells

Select compounds that inhibit neutrophil chemiluminescence for second round of screening.

2^{nd} Round Screen

Coat 384 microtitre plates with saturating concentrations of anti-CD32 Fab/F(ab′)₂ fragments

Add chemical entity selected from first to wells, over the concentration range up to 100 μM in a total volume of 5 μL.

Add 1 μL whole blood containing primed neutrophils to each well, together with HBSS and 10 μM luminol (final conc) in a total volume of 25 μL

Measure chemiluminescence generated for a period of 15 min at 37° C. Chemical entities that inhibit this activity do NOT have the required selectivity Chemical entities that inhibit neutrophil activity in the first screen, but not the second screen, are potential selective inhibitors of FcγRIIIb

1-18. (canceled)

19. A composition for the treatment of inflammatory conditions, comprising an agent which inhibits FcγRIIIb receptor activity, activation or expression.

20. The composition of claim 19, wherein the agent is a neutralising antibody, or fragment thereof, against the FCγRIIIb receptor.

21. The composition of claim 19, wherein the agent is phosphatidylinositol-phospholipase C or a functional analogue thereof.

22. The composition of claim 19, wherein the agent is an antagonist of the FcγRIIIb receptor.

23. The composition of claim 19, wherein the agent is:
(i) an agent which attenuates transmission at FcγRIIIb receptors;
(ii) an agent that increases shedding of FcγRIIIb from its GPI anchor;
(iii) an agent which reduces FcγRIIIb receptor expression and/or transcription;
(iv) an agent which inhibits synthesis or release of endogenous FcγRIIIb agonists; or
(vi) an agent which increases the rate of inactivation or metabolism of FcγRIIIb receptor agonists.

24. The composition of claim 19, wherein the composition is formulated for the treatment of Behcet's disease, ANCA associated vasculitis, systemic vasculitis, cystic fibrosis, asthma or Crohn's disease.

25. The composition of claim 19, wherein the composition is formulated for the treatment of Rheumatoid Arthritis.

26. The composition of claim 19, wherein the composition is formulated for prophylactic treatment.

27. A method for the treatment of an inflammatory condition, comprising:
administering to a subject in need of such treatment a therapeutically effective amount of an agent which inhibits FcγRIIIb receptor activity, activation or expression.

28. The method of claim 27, wherein the agent is a neutralising antibody, or fragment thereof, against the FCγRIIIb receptor.

29. The method of claim 27, wherein the agent is phosphatidylinositol-phospholipase C or a functional analogue thereof.

30. The method of claim 27, wherein the agent is an antagonist of the FcγRIIIb receptor.

31. The method of claim 27, wherein the agent is:
(i) an agent which attenuates transmission at FcγRIIIb receptors;
(ii) an agent that increases shedding of FcγRIIIb from its GPI anchor;
(iii) an agent which reduces FcγRIIIb receptor expression and/or transcription;
(iv) an agent which inhibits synthesis or release of endogenous FcγRIIIb agonists; or
(vi) an agent which increases the rate of inactivation or metabolism of FcγRIIIb receptor agonists.

32. The method of claim 27, wherein the inflammatory condition is Behcet's disease, ANCA associated vasculitis, systemic vasculitis, cystic fibrosis, asthma or Crohn's disease.

33. The method of claim 27, wherein the inflammatory condition is Rheumatoid Arthritis.

34. The method of claim 27, wherein administering to the subject in need of such treatment the therapeutically effective amount of the composition further comprises administering the composition as a prophylactic treatment.

35. A delivery system for use in a gene therapy technique, said delivery system comprising a DNA molecule encoding for a protein which directly or indirectly inhibits FcγRIIIb receptor activity, said DNA molecule being capable of being transcribed to allow the expression of said protein and thereby treat an inflammatory condition.

36. A method for the treatment of an inflammatory condition, comprising:
providing the delivery system of claim 17; and
implementing a gene therapy technique using the delivery system to treat the inflammatory condition.

37. A screening method for identifying anti-inflammatory agents, comprising:
contacting a compound with FcγRIIIb receptors; and
measuring inhibition of receptor activity or activation by said compound.

38. The method according to claim 37 comprising a 1^{st} round screen consisting of:
(1) coating a plate with saturating concentrations of anti-CD16 Fab/F(ab′)₂ fragments;
(2) priming neutrophils;
(3) adding candidate compounds to the plate;
(4) adding the primed neutrophils and luminol to the plate; and
(5) measuring chemiluminescence generated,
wherein putative anti-inflammatory agents inhibit neutrophil chemiluminescence.

39. The method according to claim 38 wherein a further 2^{nd} round screen is conducted comprising:
(6) coating plates with saturating concentrations of anti-CD32 Fab/F(ab′)₂ fragments;
(7) adding compounds selected from the 1^{st} Round Screen;
(8) adding primed neutrophils with luminol; and
(9) measuring chemiluminescence,
wherein compounds that inhibit 2^{nd} round activity do not have the required selectivity and putative anti-inflammatory agents therefore inhibit neutrophil activity in the 1^{st} Round Screen but not the 2^{nd} Round screen.

40. The screening method according to claim 38 wherein the neutrophils are isolated from blood.

41. The screening method according to claim 40 wherein whole blood is used.

42. The screening method according to claim 39 wherein the neutrophils are isolated from blood.

43. The screening method according to claim 42 wherein whole blood is used.

44. An anti-inflammatory agent identified by the screening method according to claim 37.
